(19) United States
(12) Patent Application Publication
(10) Pub. No.: US 2017/0334949 A1
(43) Pub. Date: Nov. 23, 2017

(54) REAGENT KIT AND METHOD FOR REMOVING BACTERIAL ENDOTOXIN IN BIOLOGICAL PRODUCT

(71) Applicant: Hui CHEN, NINGBO, ZHEJIANG PROVINCE (CN)

(72) Inventor: Hui CHEN, NINGBO, ZHEJIANG PROVINCE (CN)

(21) Appl. No.: 15/513,770

(22) PCT Filed: Sep. 24, 2015
(86) PCT No.: PCT/CN2015/090526

§ 371 Date: Mar. 23, 2017

(30) Foreign Application Priority Data
Sep. 24, 2014 (CN) 20140494101.9

(51) Int. Cl. C07K 1/30; C12N 15/10; C07K 14/765

(52) U.S. Cl. CPC C07K 1/30; C07K 14/765; C12N 15/1017

(57) ABSTRACT

Disclosed are a reagent kit for removing a bacterial endotoxin in a biological product, a method for using the reagent kit for removing the bacterial endotoxin in the biological product, a method for preparing an endotoxin-free biological product, and the endotoxin-free biological product thus produced. The reagent kit of the present invention comprises an anionic surfactant and a potassium salt. When in use, the anionic surfactant is fully bonded with the endotoxin in the biological product to form a conjugate, then the potassium salt is added to precipitate the conjugate, the precipitate is removed by filtration to produce a biological product solution with the endotoxin removed, and then the biological product is separated from the biological product solution to complete the process.

### BACKGROUND

### Technical Field

The present invention relates to the field of separation and purification of biological products, and particularly to a kit and a method for removing bacterial endotoxin from a biological product, and a method for preparing an endotoxin-free biological product.

### Related Art

With the wide application of modern biotechnology, the safety of biological products has been paid more and more attention, and particularly the control on pyrogens in the biological products becomes increasingly stringent. However, during the production of biological products, pyrogens are often introduced due to the factors including raw materials, production environment and personal operations etc. In the production of drugs through fermentation, the wall of the bacteria needs to be broken down to release the active substances, which will inevitably introduce a large amount of pyrogens and contaminate the active substances. Therefore, besides the efficient process control taken and the strict precautions employed to reduce the source of pyrogens in the production process, the removal of pyrogens from a biological product has become an important research subject in the art. However, heretofore there is no simple and efficient method available.

Pyrogens are pyrogenic substances that cause abnormal elevation in body temperature of homeothermic animals, and include bacterial pyrogens, endogenous high molecular weight pyrogens, endogenous low molecular weight pyrogens, and chemical pyrogens etc. The generally known “pyrogens” mainly refer to bacterial pyrogens and include metabolites of some bacteria, bacterial corpses, and endotoxin. Bacterial endotoxin is one of the major components found in the outer membrane of the cell wall of Gram-negative bacteria, which is essentially a lipopolysaccharide (LPS) and mainly composed of a polysaccharide covalently linked to Lipid A in its chemical structure. It is distinguished in that the bacterial endotoxin is not a bacterium or a metabolic product of bacteria, but a biologically active substance released after the death or disintegration of bacteria, and Lipid A is exactly the main group responsible for a variety of biological activities or toxic reactions of endotoxin. Although for different Gram-negative bacteria, the chemical composition of the LPSs is different, a Lipid A moiety is contained in each case, which means that the group is not species-specific. Therefore, the toxic reactions of the endotoxin from various species of bacteria are similar, such as fever, hemodynamic changes, disseminated intravascular coagulation, and endotoxin shock and so on. Accordingly, in order to ensure the safety of biological products in use, the endotoxin content contained therein needs to be reduced such that it falls within a safe range^{[1],[2]}. It is generally accepted that an upper endotoxin level in an injectable solution is 5 EU/kg body weight^{[3]}.

Endotoxin is very stable in chemical properties and will be failed to be destroyed under a boiling condition of 100° C., and can only be inactivated by holding at 250° C. for more than 30 minutes or 180° C. for more than 3 hours, or by soaking in strong acids or alkalis with a concentration more than 0.1 M^{[2]}. Endotoxin is hydrophobic and negatively charged under physiological conditions, and generally has a molecular weight ranging from over tens of thousands to millions of daltons. The endotoxin is generally removed by ultrafiltration, various column chromatographies (such as hydrophobic interaction chromatography, and ion exchange), affinity adsorption (polymyxin B, L-histidine, poly-L-lysine, and poly-γ-L-glutamic acid cross-linking medium) and cloud point extraction (Triton X-114), etc. A part of the endotoxin can be removed or inactivated by these methods, but there are some shortcomings. For example, ultrafiltration can only be used for small molecular weight drugs; and if the drug molecules (such as antibodies) are close to endotoxin in molecular size, effective separation cannot be achieved. The column chromatography suffers from high cost and low efficiency, and is not suitable for endotoxin removal in large-scale production. The anion exchange resin cannot be used in the separation of the same negatively charged biomolecules. The cloud point extraction (Triton X-114)^{[4]} also has fatal disadvantages, for example, the loss of active substances in the plasmids due to the need for multiple extractions during operation, and the fact that the temperature is required to be changed during treatment, and there are very fine droplets formed by Triton X-114 after phase change present in the aqueous phase, which need to be separated by centrifugation at a high speed. Therefore, the method has difficulty with respect to industrial operation and control.

In summary, it is currently an important subject in the art to develop a method that can remove endotoxin efficiently at a low cost, while the activity of a biological product is maintained to the largest extent.

### SUMMARY

In view of the technical defects existing in the prior art, a first aspect of the present invention provides a kit that is simple in operation and can effectively remove bacterial endotoxin from a biological product. The kit of the present invention comprises a potassium salt and an anionic surfactant that is insoluble in water in the presence of the potassium salt.

The anionic surfactant is one or more of sodium dodecyl sulfate, sodium deoxycholate, sodium dodecyl sulfonate, sodium s-alkyl sulfates, sodium fatty alcohol polyoxyethylene ether sulfates, sodium oleyl sulfate, N-oleoyl poly(amino acid) sodium, sodium alkylbenzene sulfonates, sodium α-olefin sulfonates, sodium alkyl sulfonates, α-sulfo monocarboxylic acid esters, fatty acid sulfoalkyl esters, succinate sulfonate, alkyl naphthalene sulfonates, sodium alkane sulfoates, sodium ligninsulfonate, sodium alkyl glyceryl ether sulfonates, and other anionic surfactants.

The anionic surfactant is sodium dodecyl sulfate, and/or sodium deoxycholate.

During the removal of endotoxin, the final concentration of the anionic surfactant after being mixed with the biological product sample from which endotoxin is intended to be removed depends on the concentration of the bacterial endotoxin in the solution. That is, the final concentration of the anionic surfactant is not particularly limited as long as it is higher than the endotoxin concentration. When the endotoxin concentration is high, the final concentration of the anionic surfactant is accordingly increased. In contrast, the anionic surfactant may be used in a low final concentration. Generally, the final concentration of the anionic surfactant is 0.1 wt % or higher, that is from 0.1 wt % to a saturation concentration.

When the biological product is a protein or DNA, the final concentration of the anionic surfactant is 0.1-10 wt %, 0.1-5 wt %, 0.1-1 wt %, 1-10 wt %, 1-5 wt %, 5-10 wt %, 0.1 wt %, 1 wt %, 5 wt %, or 10 wt %.

The potassium salt is one of potassium chloride, potassium acetate, potassium sulfate, potassium carbonate, potassium bicarbonate, potassium phosphate, potassium hydrogen phosphate, potassium dihydrogen phosphate, potassium nitrate, and other potassium salts, or a combination of two or more thereof.

The potassium salt is potassium acetate and/or potassium chloride.

During the removal of endotoxin, the final concentration of the potassium salt after being mixed with a mixed solution is higher than or equal to a concentration at which the anionic surfactant can be fully precipitated, that is, the final concentration of the potassium salt is not particularly limited as long as it can fully precipitate the anionic surfactant in the solution, where the mixed solution is composed of the anionic surfactant and the biological product sample from which endotoxin is intended to be removed.

The lowest final concentrations of the potassium salt used for precipitating different anionic surfactants are different. When sodium dodecyl sulfate is used, the final concentration of the potassium salt is 0.3 M or higher, that is from 0.3 M to a saturation concentration.

The anionic surfactant and the potassium salt may be in a solid form, or in the form of a solution (aqueous solution), and are packaged separately.

When the biological product is a protein, the final concentration of the potassium salt after being mixed with the mixed solution during the removal of endotoxin is 0.3-0.5 M, 0.3-0.55 M, 0.3-0.86 M, 0.3-1 M, 0.3-1.1 M, 0.3-1.65 M, 0.5-0.55 M, 0.5-0.86 M, 0.5-1 M, 0.5-1.1 M, 0.5-1.65 M, 0.5 M to a saturation concentration, 0.55-0.86 M, 0.55-1 M, 0.55-1.1 M, 0.55-1.65 M, 0.55 M to a saturation concentration, 0.86-1 M, 0.86-1.1 M, 0.86-1.65 M, 0.86 M to a saturation concentration, 1-1.1 M, 1-1.65 M, 1 M to a saturation concentration, 1.1-1.65 M, 1.1 M to a saturation concentration, 1.65 M to a saturation concentration, 0.3 M, 0.5 M, 0.55 M, 0.86 M, 1 M, 1.1 M, 1.65 M, or a saturation concentration.

When the biological product is DNA, the final concentration of the potassium salt after being mixed with the mixed solution during the removal of endotoxin is 0.3-0.5 M, 0.3-0.55 M, 0.3-0.69 M, 0.3-0.86 M, 0.3-1 M, 0.3-1.1 M, 0.3-1.65 M, 0.5-0.55 M, 0.5-0.69 M, 0.5-0.86 M, 0.5-1 M, 0.5-1.1 M, 0.5-1.65 M, 0.5 M to a saturation concentration, 0.55-0.69 M, 0.55-0.86 M, 0.55-1 M, 0.55-1.1 M, 0.55-1.65 M, 0.55 M to a saturation concentration, 0.69-0.86 M, 0.69-1 M, 0.69-1.1 M, 0.69-1.65 M, 0.69 M to a saturation concentration, 0.86-1 M, 0.86-1.1 M, 0.86-1.65 M, 0.86 M to a saturation concentration, 1-1.1 M, 1-1.65 M, 1 M to a saturation concentration, 1.1-1.65 M, 1.1 M to a saturation concentration, 1.65 M to a saturation concentration, 0.3 M, 0.5 M, 0.55 M, 0.69 M, 0.86 M, 1 M, 1.1 M, 1.65 M, or a saturation concentration.

A second aspect of the present invention provides a method for removing bacterial endotoxin from a biological product by using the kit mentioned above. The method comprises the following steps of: uniformly mixing a biological product containing endotoxin with the anionic surfactant solution to obtain a mixed solution, standing the resultant mixed solution, then adding the potassium salt to the solution, mixing them to completely precipitate the anionic surfactant and standing, and then centrifuging or filtering, and discarding the precipitate, thereby accomplishing the removal of endotoxin from the biological product.

The final concentration of the anionic surfactant in the mixed solution composed of the anionic surfactant and the biological product containing endotoxin is higher than that of the endotoxin.

The final concentration of the anionic surfactant is 0.1 wt % or higher, that is from 0.1 wt % to a saturation concentration.

When the biological product is a protein or DNA, the final concentration of the anionic surfactant is 0.1-10 wt %, 0.1-5 wt %, 0.1-1 wt/o, 1-10 wt %, 1-5 wt %, 5-10 wt %, 0.1 wt %, 1 wt %, 5 wt %, or 10 wt %.

The final concentration of the potassium salt after being mixed with the mixed solution is higher than or equal to the concentration at which the anionic surfactant can be sufficiently precipitated.

When sodium dodecyl sulfate is used, the final concentration of the potassium salt after being mixed with the mixed solution is 0.3 M or higher, that is from 0.3 M to a saturation concentration.

When the biological product is a protein, the final concentration of the potassium salt after being mixed with the mixed solution during the removal of endotoxin is 0.3-0.5 M, 0.3-0.55 M, 0.3-0.86 M, 0.3-1 M, 0.3-1.1 M, 0.3-1.65 M, 0.5-0.55 M, 0.5-0.86 M, 0.5-1 M, 0.5-1.1 M, 0.5-1.65 M, 0.5 M to a saturation concentration, 0.55-0.86 M, 0.55-1 M, 0.55-1.1 M, 0.55-1.65 M, 0.55 M to a saturation concentration, 0.86-1 M, 0.86-1.1 M, 0.86-1.65 M, 0.86 M to a saturation concentration, 1-1.1 M, 1-1.65 M, 1 M to a saturation concentration, 1.1-1.65 M, 1.1 M to a saturation concentration, 1.65 M to a saturation concentration, 0.3 M, 0.5 M, 0.55 M, 0.86 M, 1 M, 1.1 M, 1.65 M, or a saturation concentration.

When the biological product is DNA, the final concentration of the potassium salt after being mixed with the mixed solution during the removal of endotoxin is 0.3-0.5 M, 0.3-0.55 M, 0.3-0.69 M, 0.3-0.86 M, 0.3-1 M, 0.3-1.1 M, 0.3-1.65 M, 0.5-0.55 M, 0.5-0.69 M, 0.5-0.86 M, 0.5-1 M, 0.5-1.1 M, 0.5-1.65 M, 0.5 M to a saturation concentration, 0.55-0.69 M, 0.55-0.86 M, 0.55-1 M, 0.55-1.1 M, 0.55-1.65 M, 0.55 M to a saturation concentration, 0.69-0.86 M, 0.69-1 M, 0.69-1.1 M, 0.69-1.65 M, 0.69 M to a saturation concentration, 0.86-1 M, 0.86-1.1 M, 0.86-1.65 M, 0.86 M to a saturation concentration, 1-1.1 M, 1-1.65 M, 1 M to a saturation concentration, 1.1-1.65 M, 1.1 M to a saturation concentration, 1.65 M to a saturation concentration, 0.3 M, 0.5 M, 0.55 M, 0.69 M, 0.86 M, 1 M, 1.1 M, 1.65 M, or a saturation concentration.

The biological product is a protein or a nucleic acid.

A third aspect of the present invention provides a method for preparing an endotoxin-free biological product by using the kit above. The method comprises the steps of: uniformly mixing the biological product containing endotoxin with the anionic surfactant and standing, then adding the potassium salt and mixing uniformly until no precipitate is produced, standing and then centrifuging or filtering, collecting the filtrate that is the biological product sample solution from which the endotoxin has been removed, and then separating the biological product from the biological product sample solution from which the endotoxin has been removed, to obtain an endotoxin-free biological product.

When the biological product is a protein, the method comprises specifically the steps of:

1) adding the anionic surfactant to a protein containing endotoxin such that the final concentration of the anionic surfactant is 0.1 wt % or higher, uniformly mixing them to obtain a mixed solution, and standing the mixed solution for 5 min;

2) adding the potassium salt to the mixed solution obtained in Step 1) until no precipitate is produced, and mixing uniformly to obtain a mixed solution containing a precipitate; and standing the mixed solution for 5 min;

3) centrifuging the mixed solution containing a precipitate obtained in Step 2) at 14000 rpm for 5 min, discarding the precipitate, and collecting the supernatant; or filtering the mixed solution containing a precipitate obtained in Step 2) through a 0.45 μm PP membrane filter by centrifuging at 4000 rpm, to obtain a filtrate that is a protein sample solution from which the endotoxin has been removed; and

4) separating the protein from the protein sample solution with the removal of endotoxin obtained in Step 3) by precipitation or dialysis, to obtain an endotoxin-free protein.

When the biological product is DNA, the method comprises specifically the steps of:

1) adding the anionic surfactant to DNA containing endotoxin such that the final concentration of the anionic surfactant is 0.1 wt % or higher, uniformly mixing them to obtain a mixed solution, and standing the mixed solution for 5 min;

2) adding the potassium salt to the mixed solution obtained in Step 1) until no precipitate is produced, and mixing uniformly to obtain a mixed solution containing a precipitate; and standing the mixed solution for 5 min;

3) centrifuging the mixed solution containing a precipitate obtained in Step 2) at 14000 rpm for 5 min, discarding the precipitate, and collecting the supernatant; or filtering the mixed solution containing a precipitate obtained in Step 2) through a 0.45 μm PP membrane filter by centrifuging at 4000 rpm, to obtain a filtrate that is a DNA sample solution from which the endotoxin has been removed; and

4) adding an equal volume of isopropanol to the DNA sample solution with the removal of endotoxin obtained in Step 3), mixing uniformly and standing for 30 min at room temperature, then centrifuging at 14000 rpm for 10 min, discarding the supernatant, and washing the precipitate with 70% ethanol; centrifuging at 14000 rpm for 10 min, discarding the supernatant, washing the precipitate and centrifuging once again, and removing ethanol by air drying, to obtain an endotoxin-free DNA.

A fourth aspect of the present invention provides an endotoxin-free protein prepared by the above method, in which the endotoxin concentration is 3.5 EU/mg protein or less; and preferably the endotoxin concentration is from 1.1 to 3.5 EU/mg protein.

A fifth aspect of the present invention provides an endotoxin-free DNA prepared by the above method, in which the endotoxin concentration is 4.3 EU/mg DNA or less; and preferably the endotoxin concentration is from 1.1 to 4.3 EU/mg DNA.

Compared with the prior art, the present invention has the following beneficial effects. The endotoxin in a biological product can be conveniently and quickly removed by using the present kit. The method for removing endotoxin in the present invention has the advantages of easy operation and low cost, and has an endotoxin removal effect that is significantly superior to that of the conventional method while the biological activity of the biological product is not affected. The endotoxin concentration in the biological product prepared by the method of the present invention meets the drug standards for use in clinical, and has less loss of active substances.

### BRIEF DESCRIPTION OF THE DRAWINGS

FIG. 1 shows an endotoxin concentration-absorbance standard curve in Experimental Example 1.

### DETAILED DESCRIPTION

The present invention provides a kit for removing endotoxin from a biological product, a method for removing endotoxin from a biological product by using the same, a method for preparing an endotoxin-free biological product by using the method for removing endotoxin, and an endotoxin-free biological product prepared therefrom. The core principle of the present invention is that an anionic surfactant well binds to endotoxin, and moreover, the solubility of the anionic surfactant becomes small enough in the presence of a certain concentration of potassium ions. The addition of potassium ions can precipitate the endotoxin bound to the anionic surfactant out from an aqueous phase, thereby removing the endotoxin from a biological product. Then, the biological product with the removal of endotoxin is separated and purified from the aqueous solution by a conventional method, to obtain an endotoxin-free biological product.

The disclosure of the present invention will be described in further detail and the present invention will be further elucidated, with reference to accompanying drawings and specific embodiments; however, the present invention is not limited thereto in any way. Any changes made to the embodiments of the present invention by those skilled in the art based on the disclosure of the present invention are intended to be embraced in the protection scope as defined by the appended claims of the present invention.

The biological materials used in the examples are widely available, and any biological materials that are obtained without legal and ethical violations can be used in accordance with the instructions in the examples. The methods used are conventional methods unless otherwise particularly specified. In the examples, the material or reagent with the same name has the same contents, unless otherwise particularly specified.

### Experimental Example 1. Preparation of Biological Product Samples Before the Removal of Endotoxin

I. Plotting of Endotoxin Content-Absorbance Standard Curve from Endotoxin Standards

1. Experimental Materials

Chromogenic End-point tachypleus amebocyte lysate (TAL) kit was purchased from Xiamen Chinese Horseshoe Crab Reagent Manufactory Co., Ltd.; and the experimental equipments used are all endotoxin-free.

2. Preparation of Standard Endotoxin Solution and Plotting of Standard Curve

The procedures were performed following the instruction for use of Chromogenic End-point TAL kit provided by the Xiamen Chinese Horseshoe Crab Reagent Manufactory Co., Ltd.

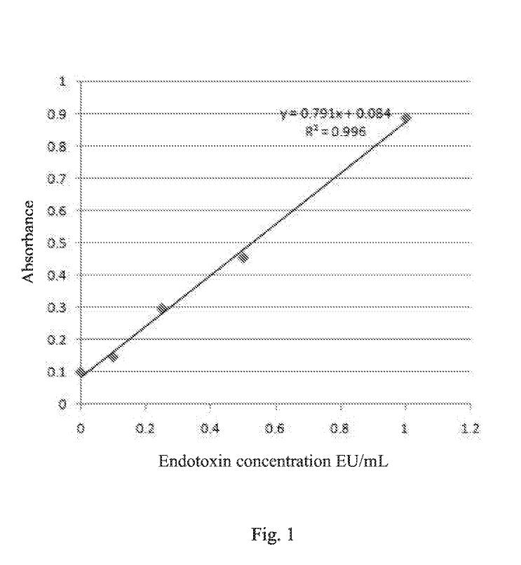

The absorbance of the endotoxin standards was determined by spectrophotometry (see table 1), and an endotoxin concentration-absorbance standard curve was obtained by plotting the absorbance on Y-axis against the endotoxin concentration on X-axis (see FIG. 1), where the equation of the standard curve was: y=0.791X+0.084(R²=0.994).

TABLE 1
Absorbance of endotoxin standards

| | Concentration of endotoxin standards EU/mL | | | | |
|---|---|---|---|---|---|
| | 0 | 0.1 | 0.25 | 0.5 | 1.0 |
| Absorbance | 0.101 | 0.148 | 0.297 | 0.455 | 0.887 |

II. Preparation of Endotoxin Solution with a High Concentration

Because the concentration of the purchased standard endotoxin solution was low, endotoxin solution with a high concentration was prepared for the sake of convenience in use during experiment.

2 mL of E. Coli DH5α was incubated overnight in a centrifuge tube, and then centrifuged at 12000 rpm for 30 s. The supernatant was discarded and the pellet was the bacteria. 200 μL of a PA solution (containing 50 mM Tris-HCl and 10 mM EDTA, pH=7.5) was added to the pellet and fully mixed uniform. Then, 200 μL of a PB solution (containing 0.2 M NaOH and 1 wt % sodium dodecyl sulfate (SDS)) was added, mixed until uniform, and stood at room temperature for 2-3 min. About 70-80 μL of a PC solution (which was a 5 M sodium acetate solution, pH=4.8) was further added, adjusted to about pH 7, and then centrifuged at 15000 rpm for 5 min. The supernatant was collected, which was endotoxin solution with a high concentration. The concentration of endotoxin in the endotoxin solution with a high concentration was about 0.5-1×10⁶ EU/mL.

III. Preparation of Biological Product Samples from which Endotoxin is Intended to be Removed

The reagents used are all common reagents commercially available.

1. Formulation of Protein Sample from which Endotoxin is Intended to be Removed

100 mg of human serum albumin (HSA) was dissolved in 1 mL of water to prepare an aqueous HSA solution. 2-10 μL of the endotoxin solution with a high concentration prepared in Section II of this experimental example was added, and mixed until uniform, to obtain a protein sample with an endotoxin concentration of about 5000-10000 EU/mL from which the endotoxin is intended to be removed. In practical operations, the range of the endotoxin concentration in the protein sample from which endotoxin is intended to be removed could be roughly controlled by increasing or decreasing the amount of the endotoxin solution with a high concentration added. If a protein sample having an endotoxin concentration falling in a low range from which endotoxin is intended to be removed is needed (for example, the endotoxin concentration is approximately 100-1000 EU/mL), this can be achieved by dilution.

2. Formulation of DNA Sample from which Endotoxin is Intended to be Removed

1 mg of PUC19 plasmid DNA was dissolved in 1 mL of water, to prepare a DNA solution. 2-10 μL of the endotoxin solution with a high concentration prepared in Section II of this experimental example was added, and mixed until uniform, to obtain a DNA sample with an endotoxin concentration of about 5000-10000 EU/mL from which the endotoxin is intended to be removed.

IV. Determination of Endotoxin Concentration in Biological Product Samples from which Endotoxin is Intended to be Removed

To compare the effect of the present invention in removing endotoxin from a biological product, the endotoxin concentration in a biological product sample from which endotoxin is intended to be removed was determined. Because the endotoxin concentration (externally added) in the biological product sample from which endotoxin is intended to be removed is too high and no linear relation exists between the endotoxin concentration and the absorbance, the biological product sample from which endotoxin is intended to be removed needs to be diluted, so that the endotoxin concentration therein falls within a range of endotoxin concentrations corresponding to the standard curve plotted in Section I of this experimental example.

1. Determination of Endotoxin Concentration in Protein Sample from which Endotoxin is Intended to be Removed

25 μL of the protein sample from which endotoxin is intended to be removed obtained in Section III of this experimental example was 20000-fold diluted with endotoxin-free water, and then the absorbance of the diluted protein sample from which endotoxin is intended to be removed was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of this experimental example, and the endotoxin concentration in the protein sample before dilution from which endotoxin is intended to be removed was obtained after calculation and conversion.

2. Determination of Endotoxin Concentration in DNA Sample from which Endotoxin is Intended to be Removed

25 μL of the DNA sample obtained in Section III of this experimental example from which endotoxin is intended to be removed was 20000-fold diluted with endotoxin-free water, and then the absorbance of the diluted DNA sample from which endotoxin is intended to be removed was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of this experimental example, and the endotoxin concentration in the DNA sample from which endotoxin is intended to be removed was calculated.

### Example 1. Removal of Endotoxin from Biological Product Samples from which Endotoxin is Intended to be Removed

1) An anionic surfactant was added to a biological product sample obtained through the method in Section III of Experimental Example 1 from which endotoxin is intended to be removed, such that the final concentration of the anionic surfactant was not less than 0.1 wt % (where the final concentration of the anionic surfactant depends on the endotoxin concentration, needs to be increased accordingly with the increase of the endotoxin concentration, and is generally 0.1 wt % or higher, and preferably ranges from 5 to 10 wt %). After being fully mixed until uniform, a mixed solution was obtained, and stood for 5 min. The anionic surfactant may be one or more selected from sodium dodecyl sulfate (SDS), sodium deoxycholate (SDC), sodium s-alkyl sulfates, sodium fatty alcohol polyoxyethylene ether sulfates, sodium oleyl sulfate, N-oleoyl poly(amino acid) sodium, sodium alkylbenzene sulfonates, sodium α-olefin sulfonates, sodium alkyl sulfonates, α-sulfo monocarboxylic acid esters, fatty acid sulfoalkyl esters, succinate sulfonate, alkyl naphthalene sulfonates, sodium alkane sulfoates, sodium ligninsulfonate, sodium alkyl glyceryl ether sulfonates, and other anionic surfactants that are insoluble in water in the presence of a potassium salt. A solid anionic surfactant may be used directly, or an aqueous solution of an anionic surfactant may also be used.

2) A potassium salt was added to the mixed solution obtained in Step 1) until no precipitate was produced, and mixed until uniform, to obtain a mixed solution containing a precipitate. Then the mixed solution was stood for 5 min. The final concentration of the potassium salt in the mixed solution obtained in Step 1) is not particularly limited, provided that the anionic surfactant can be precipitated completely. The potassium salt may be selected from potassium chloride (KCl), potassium acetate (KAc), potassium carbonate (K₂CO₃), potassium bicarbonate (KHCO₃), potassium phosphate (K₃PO₄), potassium hydrogen phosphate (K₂HPO₄), potassium dihydrogen phosphate (KH₂PO₄) or potassium sulfate (K₂SO₄), and other commonly used potassium salt, which may be in a solid form, or in the form of an aqueous solution.

3) The mixed solution containing a precipitate obtained in Step 2) was centrifuged at 14000 rpm for 5 min, the precipitate was discarded, and the supernatant was collected; or the mixed solution containing a precipitate was filtered through a 0.45 μm PP membrane filter by centrifuging at 4000 rpm. The filtrate was collected, which was the biological product sample solution from which the endotoxin had been removed.

Hereinafter, the method for removing endotoxin provided in the present invention is described in detailed where a protein and DNA are used as examples.

### Experimental Example 2. Interference Test on Endotoxin Detection

To eliminate the interference of the absorbance of a biological product on that of the endotoxin, an interference test was carried out, so as to screen out a concentration of the biological product that has the minimum interference on endotoxin detection. The operation procedure of the interference test was performed following “Interference Test of Test Product” in instruction for use of Chromogenic End-point TAL kit provided by the Xiamen Chinese Horseshoe Crab Reagent Manufactory Co., Ltd.

1. Interference Test of Protein Sample Solution on Endotoxin Detection

Because a potassium salt needs to be added in removal of endotoxin from a protein sample solution, the protein sample solution needs to be diluted in a certain fold to eliminate the interference before it is determined.

A protein sample solution from which the endotoxin had been removed, for example, the protein sample solution (pH 7-8) obtained in Step 3) in Example 1 from which the endotoxin had been removed was 25-, 50- and 100-fold diluted respectively, to obtain three samples for interference test. 0.5 mL of 1 EU/mL endotoxin solution was added to the three samples for interference test respectively. The samples for interference test were determined following the instruction for use of the kit above, and then the experimental value of the endotoxin concentration was calculated by using the standard curve shown in FIG. 1. The test results are shown in Table 2.

TABLE 2
Results of interference test of K⁺ ion concentration
in protein sample solution on endotoxin detection

| Composition of samples for | | Amount of | Experimental value |
|---|---|---|---|
| protein interference test | | endotoxin | of endotoxin |
| Dilution | Endotoxin | added | concentration |
| factor | (EU/mL) | (EU/mL) | (EU/mL) |
| 25 | 0.5 | 0.5 | 0 (not |
| | | | detected) |
| 50 | 0.5 | 0.5 | 0.45 |
| 100 | 0.5 | 0.5 | 0.55 |

It can be seen from the test results in Table 2 that when the protein sample solution is diluted by 100 folds or higher, the interference on detection is eliminated. Therefore, before subsequent determination of the endotoxin concentration in the protein sample solution, the sample solution is over 100-fold diluted with endotoxin-free water and then determined.

2. Interference Test of DNA Sample Solution on Endotoxin Detection

Because the extraction of solid DNA from a DNA sample solution was much easier than the protein extraction, when the endotoxin concentration in a DNA sample solution was detected, equal volume of isopropanol was added to the DNA sample solution from which endotoxin had been removed, for example, the DNA sample solution obtained in Step 3) in Example 1 from which the endotoxin had been removed, mixed until uniform and stood at room temperature for 30 min, and then centrifuged at 14000 rpm for 10 min. The supernatant was discarded, and the pellet was washed with a suitable amount of 70% ethanol, and further centrifuged at 14000 rpm for 10 min. The supernatant was discarded, the pellet was washed and centrifuged once again, and the ethanol was removed by air drying, to obtain an endotoxin-free DNA.

The obtained endotoxin-free DNA was dissolved in endotoxin-free water, and then the detection for endotoxin concentration was conducted. In this way, the interference from other substances was eliminated. To eliminate the influence of DNA per se on endotoxin detection, the DNA needed to be diluted to a certain concentration before it is determined, so as to eliminate the interference.

The endotoxin-free DNA was formulated with endotoxin-free water to provide five DNA solutions with a DNA concentration of 0.2 mg/mL, 0.4 mg/mL, 0.6 mg/mL, 0.8 mg/mL, and 1.0 mg/mL. 0.5 mL of 1 EU/mL endotoxin standard was added to 0.5 mL of the five DNA solutions respectively, to obtain five samples for DNA interference test. The samples for interference test were determined following the instruction for use of the kit above, and then the experimental value of the endotoxin concentration was calculated by using the standard curve shown in FIG. 1. The test results are shown in Table 3.

TABLE 3
Results of interference test of DNA
concentration on endotoxin detection

| | | |
|---|---|---|
| Final concentration of | | |
| materials in samples for | Amount of | Experimental value |
| DNA interference test | endotoxin | of endotoxin |

| DNA | Endotoxin | added | concentration |
|---|---|---|---|
| (mg/mL) | (EU/mL) | (EU/mL) | (EU/mL) |
| 0.1 | 0.5 | 0.5 | 0.49 |
| 0.2 | 0.5 | 0.5 | 0.54 |
| 0.3 | 0.5 | 0.5 | 0.51 |
| 0.4 | 0.5 | 0.5 | 0.21 |
| 0.5 | 0.5 | 0.5 | 0.13 |

It can be seen from the test results in Table 3 that when the DNA concentration is less than 0.3 mg/mL, the interference on detection is eliminated. Therefore, before subsequent determination of the endotoxin concentration, the sample is diluted with endotoxin-free water to give a DNA concentration less than 0.3 mg/mL.

### Example 2. Removal of Endotoxin from Protein Sample by Using Aqueous SDS Solution and Aqueous Potassium Chloride Solution

1) An aqueous sodium dodecyl sulfate (SDS) solution was added to 0.5 mL of a protein sample (in which the endotoxin concentration was about 8000 EU/mL) obtained through the method in Section III of Experimental Example 1 from which endotoxin was intended to be removed, and mixed until uniform, to obtain a mixed solution with a final volume of 1 mL. Then, the mixed solution was stood for 5 min. The final concentration of SDS in the mixed solution was 1 wt %, 5 wt %, and 10 wt % respectively.

2) 0.4 mL of 3 M potassium chloride solution (where pH=7.5, 3 M means 3 mol/L, and the final concentration of potassium chloride in the mixed solution was 0.86 M) was added to the mixed solution obtained in Step 1), and mixed until uniform, to obtain a mixed solution containing a precipitate. Then, the mixed solution containing a precipitate was stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a protein sample solution from which the endotoxin had been removed.

Detection of Endotoxin Concentration

50 μL of the protein sample solution obtained in Step 3) from which endotoxin had been removed was 100-fold diluted with endotoxin-free water, and then the absorbance of the diluted protein sample solution from which the endotoxin had been removed was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the protein sample solution before dilution from which the endotoxin had been removed was obtained after calculation and conversion. The results are 3.5 EU/mg, 2.5 EU/mg, and 1.1 EU/mg respectively. The protein concentration in the protein sample solution from which the endotoxin had been removed was detected at 280 nm, and found to be 91 mg/mL (recovery rate: 91%), 86 mg/mL (recovery rate: 86%), and 79 mg/mL (recovery rate: 79%) respectively.

It can be seen from the results obtained in this example that the higher the final concentration of the anionic surfactant in Step 1) is, the better the endotoxin removal effect will be. When the final concentration of the anionic surfactant reaches 1 wt % after being mixed with the protein sample from which endotoxin is intended to be removed, the endotoxin concentration in the protein sample can be reduced to meet the drug standards for use in clinical (where the endotoxin concentration is required to be less than 5-10 EU/mg (in which the endotoxin concentration in recombinant human growth hormone needs to be less than 5 EU/mg; and the endotoxin concentration in recombinant human insulin needs to be less than 10 EU/mg, Pharmacopoeia of the People's Republic of China, 2005 Edition, Part II, Page 495)), and the recovery rate can be reached to 80% basically, indicating that the method according to the present invention had a good effect in the removal of endotoxin from a protein sample, with the protein loss being small.

### Example 3. Removal of Endotoxin from Protein Sample by Using Aqueous SDS Solution and Aqueous Potassium Acetate Solution

1) 0.5 mL of 10 wt % aqueous sodium dodecyl sulfate (SDS) solution was added to 0.5 mL of a protein sample (in which the endotoxin concentration was about 8000 EU/mL) obtained through the method in Section III of Experimental Example 1 from which endotoxin was intended to be removed such that the final concentration of SDS was 5 wt %, and mixed until uniform, to obtain a mixed solution. Then the mixed solution was stood for 5 min.

2) 0.1 mL, 0.2 mL, 0.5 mL, and 1 mL of 3.3 M potassium acetate solution (pH=7.5) was added to the mixed solution obtained in Step 1) respectively such that the final concentration of potassium acetate in the mixed solution was 0.3 M, 0.55 M, 1.1 M, and 1.65 M respectively, and mixed until uniform, to obtain a mixed solution containing a precipitate. Then, the mixed solution containing a precipitate was stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a protein sample solution from which the endotoxin had been removed.

Detection of Endotoxin Concentration

50 μL of the protein sample solution obtained in Step 3) from which endotoxin had been removed was 100-fold diluted with endotoxin-free water, and then the absorbance of the diluted protein sample solution from which the endotoxin had been removed was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the protein sample solution before dilution from which the endotoxin had been removed was obtained after calculation and conversion. The results are 1.5 EU/mg, 1.9 EU/mg, 1.2 EU/mg, and 1.4 EU/mg respectively. The recovery rate of protein after endotoxin removal can be reached to 80% or above in each case.

The results obtained in this example show that the variation in the concentration of the precipitating agent, i.e. the potassium salt, has no influence on the removal of endotoxin in the protein sample. Under the conditions provided in this example, the concentration of the precipitating agent is not particularly limited, provided that the precipitation is complete and the endotoxin concentration in the protein sample can be reduced to meet the drug standards for use in clinical. In this case, the final concentration of the precipitating agent is not less than 0.3 M. The recovery rate of protein after endotoxin removal can be reached to 80% or above in each case, indicating that the effect of endotoxin removal from a protein sample is good and the protein recovery rate is high, when the final concentration of the precipitating agent is not less than 0.3 M.

### Example 4. Removal of Endotoxin from Protein Sample by Using Aqueous SDC Solution and Aqueous Potassium Acetate Solution

1) An aqueous sodium deoxycholate (SDC) solution was added to 0.5 mL of a protein sample (in which the endotoxin concentration was about 500 EU/mL) obtained through the method in Section III of Experimental Example 1 from which endotoxin was intended to be removed, and mixed until uniform, to obtain a mixed solution with a final volume of 1 mL. Then, the mixed solution was stood for 5 min. The final concentration of SDC in the mixed solution was 0.1 wt %.

2) 0.4 mL of 3 M potassium acetate solution (pH=7.5) was added to the mixed solution obtained in Step 1), and mixed until uniform, to obtain a mixed solution containing a precipitate. Then, the mixed solution containing a precipitate was stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a protein sample solution from which the endotoxin had been removed.

Detection of Endotoxin Concentration

50 μL of the protein sample solution obtained in Step 3) from which endotoxin had been removed was 100-fold diluted with endotoxin-free water, and then the absorbance of the diluted protein sample solution from which the endotoxin had been removed was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the protein sample solution before dilution from which the endotoxin had been removed was obtained after calculation and conversion. The result is 1.5 EU/mg.

### Example 5. Removal of Endotoxin from Protein Sample by Using Aqueous SDC Solution and Aqueous Potassium Acetate Solution

1) 0.5 mL of 10 wt % aqueous sodium deoxycholate (SDC) solution was added to 0.5 mL of a protein sample (in which the endotoxin concentration was about 8000 EU/mL) obtained through the method in Section III of Experimental Example 1 from which endotoxin was intended to be removed such that the final concentration of SDC was 5 wt %, and mixed until uniform, to obtain a mixed solution. Then the mixed solution was stood for 5 min.

2) 0.4 mL of 3 M potassium acetate solution (pH=7.5) was added to the mixed solution obtained in Step 1) and mixed until uniform, to obtain a mixed solution containing a precipitate. Then, the mixed solution containing a precipitate was stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a protein sample solution from which the endotoxin had been removed.

Detection of Endotoxin Concentration

50 μL of the protein sample solution obtained in Step 3) from which endotoxin had been removed was 100-fold diluted with endotoxin-free water, and then the absorbance of the diluted protein sample solution from which the endotoxin had been removed was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the protein sample solution before dilution from which the endotoxin had been removed was obtained after calculation and conversion. The result is 1.5 EU/mg.

It can be seen from the combination of the results obtained in Example 2 and this example that SDS and SDC are comparable in the effects of endotoxin removal from protein samples, and there is no significant difference therebetween.

### Example 6. Removal of Endotoxin from Protein Sample by Using Solid SDS and Solid Potassium Chloride

1) 0.3 g of solid sodium dodecyl sulfate (SDS) was added to 0.5 mL of a protein sample (in which the endotoxin concentration was about 8000 EU/mL) obtained through the method in Section III of Experimental Example 1 from which endotoxin was intended to be removed, heated to 45° C. and maintained at this temperature for 10 min. Solid SDS was separated out after cooling to room temperature. A mixed solution was obtained, which was then stood for 5 min.

2) 0.3 g of solid potassium chloride was added to the mixed solution obtained in Step 1), and mixed fully, to well dissolve the solid potassium chloride. A mixed solution containing a precipitate (comprising solid potassium chloride) was obtained, which was then stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuged at 4000 rpm, to obtain a filtrate that was a protein sample solution from which the endotoxin had been removed.

Detection of Endotoxin Concentration

50 μL of the protein sample solution obtained in Step 3) from which endotoxin had been removed was 100-fold diluted with endotoxin-free water, and then the absorbance of the diluted protein sample solution from which the endotoxin had been removed was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the protein sample solution before dilution from which the endotoxin had been removed was obtained after calculation and conversion. The result was 1.2 EU/mg.

The result obtained in this example shows that the final concentration of SDS reaches a saturation concentration by using solid SDS in Step 1), and the final concentration of the potassium salt also reaches a saturation concentration by using a solid potassium salt in Step 2); under these conditions, endotoxin can also be removed effectively; and the effect is comparable to that obtained when an aqueous SDS solution and an aqueous potassium salt solution are used, and there is no significant difference therebetween. Moreover, when solid SDS is used, whether the final concentration of SDS reaches a saturation concentration and whether the potassium salt is in the solid form or in the form of an aqueous solution, the endotoxin removal effect is not affected. Likewise, when a solid potassium salt is used, whether the final concentration of the potassium salt reaches a saturation concentration and whether the SDS is in the solid form or in the form of an aqueous solution, the endotoxin removal effect is not affected.

In summary, for a protein sample, the endotoxin concentration in the protein sample can be reduced to meet the drug standards for use in clinical (where the endotoxin concentration is required to be less than 5-10 EU/mg (in which the endotoxin concentration in recombinant human growth hormone needs to be less than 5 EU/mg; and the endotoxin concentration in recombinant human insulin needs to be less than 10 EU/mg, Pharmacopoeia of the People's Republic of China, 2005 Edition, Part II, Page 495)), as long as the final concentration of the anionic surfactant after being mixed with the protein sample from which endotoxin is intended to be removed is not less than 0.1 wt %, and the potassium salt is mixed with the mixed solution obtained in Step 1) until no precipitate is produced. The type of the anionic surfactant or potassium salt has no influence on the removal effect. Particularly, the variation in the final concentration of the precipitating agent, i.e. the potassium salt, has no influence on the removal of endotoxin in the protein sample, since the endotoxin concentration in the protein sample can be reduced to meet the drug standards for use in clinical provided that the precipitation is complete. Similar experimental results can be achieved with other types of anionic surfactants and other types of potassium salts, which are not enumerated herein.

### Example 7. Protein Separation after Endotoxin Removal

The protein in the protein sample solution obtained in Step 3) from which the endotoxin had been removed was separated by conventional precipitation or dialysis (for example, the method mentioned in Purification of Human Serum Albumin from Plasma with the Combination of Hydrophobic Interaction Chromatography and Cold Ethanol Precipitation, Chinese Journal of Biotechnology, Vol. 20, No. 6, Pages 943-947), to obtain a protein with the removal of endotoxin, or an endotoxin-free protein.

### Example 8. Removal of Endotoxin from DNA Sample by Using Aqueous SDS Solution and Aqueous Potassium Acetate Solution

1) An aqueous sodium dodecyl sulfate (SDS) solution was added to 0.5 mL of a DNA sample (in which the DNA concentration was 1 mg/mL, and the endotoxin concentration was about 8000 EU/mL) obtained through the method in Section III of Experimental Example 1 from which endotoxin was intended to be removed, and mixed until uniform, to obtain a mixed solution with a final volume of 1 mL. Then, the mixed solution was stood for 5 min. The final concentration of the aqueous SDS solution in the mixed solution was 1 wt %, 5 wt %, and 10 wt % respectively.

2) 0.3 mL of 3 M potassium acetate solution (where pH=5, and the final concentration of potassium acetate in the mixed solution was 0.69 M) was added to the mixed solution obtained in Step 1), and mixed until uniform, to obtain a mixed solution containing a precipitate. Then, the mixed solution containing a precipitate was stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a DNA sample solution from which the endotoxin had been removed.

DNA Separation after Endotoxin Removal

An equal volume of isopropanol was added to the DNA sample solution with the removal of endotoxin obtained in Step 3), mixed until uniform, stood at room temperature for 30 min, and then centrifuged at 14000 rpm for 10 min. The supernatant was discarded, and the precipitate was washed with a suitable amount of 70% ethanol, and further centrifuged at 14000 rpm for 10 min. The supernatant was discarded, the precipitate was washed and centrifuged once again, and then ethanol was removed by air drying, to obtain 5 DNA with the removal of endotoxin or an endotoxin-free DNA.

Detection of Endotoxin Concentration

The endotoxin-free DNA was dissolved in endotoxin-free water, such that the DNA concentration was less than 0.3 mg/mL. Then the absorbance of the endotoxin-free DNA was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the endotoxin-free DNA was calculated. The results are 4.3 EU/mg, 2.1 EU/mg, and 1.1 EU/mg respectively. The DNA concentration was detected at 260 nm and found to be 0.87 mg/mL (recovery rate: 87%), 0.82 mg/mL (recovery rate: 82%), and 0.77 mg/mL (recovery rate: 77%) respectively.

The results obtained in this example show that similar to the results obtained with a protein, when the biological product is DNA, the higher the final concentration of the anionic surfactant in Step 1) is, the better the endotoxin removal effect will be. When the final concentration of the anionic surfactant reaches 1 wt % after being mixed with the DNA sample from which endotoxin is intended to be removed, the endotoxin concentration in the DNA sample can be reduced to meet the drug standards for use in clinical (where the endotoxin content is required to be not higher than 10 EU/mg, according to 9: Pyrogenic test in Section 5 of Technical Guidelines for Pre-Clinical Research of Preventive DNA Vaccines issued by the State Food and Drug Administration on Mar. 20, 2003, which is mainly used for detecting the presence of pyrogenic substances in a product, and the bacterial endotoxin can be detected with tachypleus amebocyte lysate (TAL)), and the DNA recovery rate is reached to 75% or higher in each case, indicating that the method according to the present invention has a good effect in the removal of endotoxin from a DNA sample, with the DNA recovery rate being high.

### Example 9. Removal of Endotoxin from DNA Sample by Using Aqueous SDC Solution and Aqueous Potassium Acetate Solution

1) 0.5 mL of 10 wt % aqueous sodium deoxycholate (SDC) solution was added to 0.5 mL of a DNA sample (in which the DNA concentration was 1 mg/mL, and the endotoxin concentration was about 8000 EU/mL) obtained through the method in Section III of Experimental Example 1 from which endotoxin was intended to be removed such that the final concentration of SDC was 5 wt %, and mixed until uniform, to obtain a mixed solution. Then the mixed solution was stood for 5 min.

2) 0.1 mL, 0.2 mL, 0.5 mL, and 1 mL of 3.3 M potassium acetate solution (pH=5) was added to the mixed solution obtained in Step 1) respectively, such that the final concentration of potassium acetate in the mixed solution was 0.3 M, 0.55 M, 1.1 M, and 1.65 M respectively, and mixed until uniform, to obtain a mixed solution containing a precipitate. Then, the mixed solution containing a precipitate was stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a DNA sample solution from which the endotoxin had been removed.

DNA Separation after Endotoxin Removal

An equal volume of isopropanol was added to the DNA sample solution with the removal of endotoxin obtained in Step 3), mixed until uniform, stood at room temperature for 30 min, and then centrifuged at 14000 rpm for 10 min. The supernatant was discarded, and the precipitate was washed with a suitable amount of 70% ethanol, and further centrifuged at 14000 rpm for 10 min. The supernatant was discarded, the precipitate was washed and centrifuged once again, and then ethanol was removed by air drying, to obtain DNA with the removal of endotoxin or an endotoxin-free DNA.

Detection of Endotoxin Concentration

The endotoxin-free DNA was dissolved in endotoxin-free water, such that the DNA concentration was less than 0.3 mg/mL. Then the absorbance of the endotoxin-free DNA sample was determined following the determination method as described in the instruction for use of Chromogenic End-point TAL kit provided by the Xiamen Chinese Horseshoe 5 Crab Reagent Manufactory Co., Ltd. Then, the obtained absorbance value was substituted into the equation of standard curve obtained through the method in Section I of Experimental Example 1, and the endotoxin concentration in the endotoxin-free DNA was calculated. The results are 1.2 EU/mg, 1.5 EU/mg, 1.8 EU/mg, and 1.4 EU/mg respectively.

The results obtained in this example show that the variation in the concentration of the precipitating agent, i.e. the potassium salt, has no influence on the removal of endotoxin in the DNA sample. Under the conditions provided in this example, the concentration of the precipitating agent is not particularly limited, provided that the precipitation is complete and the endotoxin concentration in the protein sample can be reduced to meet the drug standards for use in clinical. In this case, the final concentration of the precipitating agent is not less than 0.3 M. The recovery rate of DNA after endotoxin removal can be reached to 75% or above in each case, indicating that the effect of endotoxin removal from a DNA sample is good and the DNA recovery rate is high, when the final concentration of the precipitating agent is not less than 0.3 M.

### Example 10. Removal of Endotoxin from DNA Sample by Using Aqueous SDS Solution and Aqueous Potassium Acetate Solution

1) An aqueous sodium dodecyl sulfate (SDS) solution was added to 0.5 mL of a DNA sample (in which the DNA concentration was 1 mg/mL, and the endotoxin concentration was about 500 EU/mL) obtained through the method in Section III of Experimental Example 1 from which endotoxin was intended to be removed, and mixed until uniform, to obtain a mixed solution with a final volume of 1 mL. Then, the mixed solution was stood for 5 min. The final concentration of the aqueous SDS solution in the mixed solution was 0.1 wt %.

2) 0.3 mL of 3 M potassium acetate solution (where pH=5, and the final lean Copy concentration of potassium acetate in the mixed solution was 0.69 M) was added to the mixed solution obtained in Step 1), and mixed until uniform, to obtain a mixed solution containing a precipitate. Then, the mixed solution containing a precipitate was stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a DNA sample solution from which the endotoxin had been removed.

Detection of Endotoxin Concentration

The endotoxin-free DNA was dissolved in endotoxin-free water, such that the DNA concentration was less than 0.3 mg/mL. Then the absorbance of the endotoxin-free DNA sample was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the endotoxin-free DNA was calculated. The result is 2 EU/mg.

### Example 11. Removal of Endotoxin from DNA Sample by Using Aqueous SDS Solution and Aqueous Potassium Chloride Solution

1) 10 wt % aqueous sodium dodecyl sulfate (SDS) solution was added to 0.5 mL of a DNA sample (in which the DNA concentration was 1 mg/mL, and the endotoxin concentration was about 8000 EU/mL) obtained through the method in Section III of Experimental Example 1 from which endotoxin was intended to be removed, and mixed until uniform to obtain a mixed solution with a final volume of 1 mL. Then the mixed solution was stood for 5 min. The final concentration of SDS in the mixed solution was 1 wt % (since the volume of the aqueous SDS solution was inadequate, water was added up to 1 mL).

2) 0.2 mL of 3 M potassium chloride solution (formulated with 10 mM Tris-HCl solution, pH=7.5) was added to the mixed solution obtained in Step 1) such that the final concentration of potassium chloride in the mixed solution was 0.5 M, and mixed until uniform, to obtain a mixed solution containing a precipitate. Then, the mixed solution containing a precipitate was stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a DNA sample solution from which the endotoxin had been removed.

DNA Separation after Endotoxin Removal

An equal volume of isopropanol was added to the DNA sample solution with the removal of endotoxin obtained in Step 3), mixed until uniform, stood at room temperature for 30 min, and then centrifuged at 14000 rpm for 10 min. The supernatant was discarded, and the precipitate was washed with a suitable amount of 70% ethanol, and further centrifuged at 14000 rpm for 10 min. The supernatant was discarded, the precipitate was washed and centrifuged once again, and then ethanol was removed by air drying, to obtain DNA with the removal of endotoxin or an endotoxin-free DNA.

Detection of Endotoxin Concentration

The endotoxin-free DNA was dissolved in endotoxin-free water, such that the DNA concentration was less than 0.3 mg/mL. Then the absorbance of the endotoxin-free DNA sample was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the endotoxin-free DNA was calculated. The result is 1.9 EU/mg.

It can be seen from the combination of the results obtained in Example 10 and this example that when used as a precipitating agent, potassium chloride and potassium acetate are comparable in the effects of removing endotoxin from DNA samples, and there is no significant difference therebetween.

### Example 12. Removal of Endotoxin from DNA Sample by Using Aqueous SDC Solution and Aqueous Potassium Acetate Solution

1) 0.5 mL of 10 wt % aqueous sodium deoxycholate (SDC) solution was added to 0.5 mL of a DNA sample (in which the DNA concentration was 1 mg/mL, and the endotoxin concentration was about 8000 EU/mL) obtained through the method in Section III of Experimental Example 1 from which endotoxin was intended to be removed such that the final concentration of SDC was 5 wt %, and mixed until uniform, to obtain a mixed solution. Then the mixed solution was stood for 5 min.

2) 0.4 mL of 3 M potassium acetate solution (pH=5) was added to the mixed solution obtained in Step 1) such that the final concentration of potassium acetate in the mixed solution was 0.86 M, and mixed until uniform, to obtain a mixed solution containing a precipitate. Then, the mixed solution containing a precipitate was stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a DNA sample solution from which the endotoxin had been removed.

DNA Separation after Endotoxin Removal

An equal volume of isopropanol was added to the DNA sample solution with the removal of endotoxin obtained in Step 3), mixed until uniform, stood at room temperature for 30 min, and then centrifuged at 14000 rpm for 10 min. The supernatant was discarded, and the precipitate was washed with a suitable amount of 70% ethanol, and further centrifuged at 14000 rpm for 10 min. The supernatant was discarded, the precipitate was washed and centrifuged once again, and then ethanol was removed by air drying, to obtain DNA with the removal of endotoxin or an endotoxin-free DNA.

Detection of Endotoxin Concentration

The endotoxin-free DNA was dissolved in endotoxin-free water, such that the DNA concentration was less than 0.3 mg/mL. Then the absorbance of the endotoxin-free DNA sample was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the endotoxin-free DNA was calculated. The result is 1.2 EU/mg.

It can be seen from the combination of the results obtained in Example 11 and this example that SDS and SDC are comparable in the effects of removing endotoxin from DNA samples, and there is no significant difference therebetween.

### Example 13. Removal of Endotoxin from DNA Sample by Using Solid SDS and Solid Potassium Chloride

1) 0.3 g of solid sodium dodecyl sulfate (SDS) was added to 0.5 mL of a DNA sample (in which the DNA concentration was 1 mg/mL and the endotoxin concentration was about 8000 EU/mL) obtained through the method in Section III of Experimental Example 1 from which endotoxin was intended to be removed, heated to 45° C. and maintained at this temperature for 10 min. Solid SDS was separated out after cooling to room temperature. A mixed solution was obtained, which was then stood for 5 min.

2) 0.3 g of solid potassium chloride was added to the mixed solution obtained in Step 1), and mixed fully, to well dissolve the solid potassium chloride. A mixed solution containing a precipitate (comprising undissolved solid potassium chloride) was obtained, which was then stood for 5 min.

3) The mixed solution containing a precipitate obtained in Step 2) was centrifuged at 14000 rpm for 5 min, the precipitate was discarded, and the supernatant was collected; or the mixed solution containing a precipitate obtained in Step 2) was filtered through a 0.45 μm PP membrane filter by centrifuging at 4000 rpm, to obtain a filtrate that was a DNA sample solution from which the endotoxin had been removed.

DNA Separation after Endotoxin Removal

An equal volume of isopropanol was added to the DNA sample solution with the removal of endotoxin obtained in Step 3), mixed until uniform, stood at room temperature for 30 min, and then centrifuged at 14000 rpm for 10 min. The supernatant was discarded, and the precipitate was washed with a suitable amount of 70% ethanol, and further centrifuged at 14000 rpm for 10 min. The supernatant was discarded, the precipitate was washed and centrifuged once again, and then ethanol was removed by air drying, to obtain DNA with the removal of endotoxin or an endotoxin-free DNA.

Detection of Endotoxin Concentration

The endotoxin-free DNA was dissolved in endotoxin-free water, such that the DNA concentration was less than 0.3 mg/mL. Then the absorbance of the endotoxin-free DNA sample was determined following the determination method as described in the instruction for use of Chromogenic End-point TAL kit provided by the Xiamen Chinese Horseshoe Crab Reagent Manufactory Co., Ltd. Then, the obtained absorbance value was substituted into the equation of standard curve obtained through the method in Section I of Experimental Example 1, and the endotoxin concentration in the endotoxin-free DNA was calculated. The result is 1.1 EU/mg.

The result obtained in this example shows that the final concentration of SDS reaches a saturation concentration by using solid SDS in Step 1), and the final concentration of the potassium salt also reaches a saturation concentration by using a solid potassium salt in Step 2); under these conditions, endotoxin can also be removed effectively; and the effect is comparable to that obtained when an aqueous SDS solution and an aqueous potassium salt solution are used, and there is no significant difference therebetween. Moreover, when solid SDS is used, whether the final concentration of SDS reaches a saturation concentration and whether the potassium salt is in the solid form or in the form of an aqueous solution, the endotoxin removal effect is not affected. Likewise, when a solid potassium salt is used, whether the final concentration of the potassium salt reaches a saturation concentration and whether the SDS is in the solid form or in the form of an aqueous solution, the endotoxin removal effect is not affected.

In summary, for a DNA sample, the endotoxin concentration in the DNA sample can be reduced to meet the drug standards for use in clinical (where the endotoxin content is required to be not higher than 10 EU/mg, according to 9: Pyrogenic test in Section 5 of Technical Guidelines for Pre-Clinical Research of Preventive DNA Vaccines issued by the State Food and Drug Administration on Mar. 20, 2003, which is mainly used for detecting the presence of pyrogenic substances in a product, and the bacterial endotoxin can be detected with tachypleus amebocyte lysate (TAL)), as long as the final concentration of the anionic surfactant after being mixed with the DNA sample from which endotoxin is intended to be removed is not less than 0.1 wt %, and the potassium salt is mixed with the mixed solution obtained in Step 1) until no precipitate is produced. The type of the anionic surfactant or potassium salt has no influence on the removal effect. Particularly, the variation in the final concentration of the precipitating agent, i.e. the potassium salt, has no influence on the removal of endotoxin from the DNA sample, since the endotoxin concentration in the protein sample can be reduced to meet the drug standards for use in clinical provided that the precipitation is complete. Similar experimental results can be achieved with other types of anionic surfactants and other types of potassium salts, which are not enumerated herein.

Since RNA and DNA are similar in nature, endotoxin in RNA can also be removed by using the methods above.

### Experimental Example 3. Influence of Different Concentrations of Biological Product in Samples on Effect of Endotoxin Removal

To investigate the influence of the concentration of a biological product in the biological product sample on removal of endotoxin from the sample, multiple groups of parallel experiments for removing endotoxin from a biological product sample were conducted following the methods as described in Experimental Example 1 and Example 1, where samples of different biological products were used (in which if the biological product was Human serum albumin, the biological product sample was a protein sample; and if the biological product was DNA, the biological product sample was a DNA sample). The experimental results are shown in Tables 4 and 5 (endotoxin concentration before removal and endotoxin concentration after removal refer to the endotoxin concentrations in the biological product sample before and after endotoxin removal, and where a dilution 5 operation is performed, the concentration has been converted into the concentration before dilution).

The anionic surfactant in Step 1) of Example 1 is SDS with the final concentration of 5 wt %; and the potassium salt in Step 2) is potassium acetate with the final concentration of 0.7 M.

Recovery rate of the DNA sample: DNA was separated and extracted following a conventional method from the DNA sample solution from which the endotoxin had been removed, weighed and compared with the weight of DNA added to the DNA sample from which endotoxin was intended to be removed. Recovery rate of the protein sample: the protein concentration in the protein sample solution from which endotoxin had been removed was detected by a UV spectrophotometer at 280 nm and then compared with the protein concentration in the protein sample from which endotoxin was intended to be removed.

TABLE 4
Influence of different concentrations of DNA in
DNA sample on endotoxin removal from DNA sample

| | DNA concentration in DNA sample, mg/mL | | | | |
|---|---|---|---|---|---|
| | 0.25 | 0.5 | 1 | 1.5 | 2 |
| Endotoxin concentration | 8000 | 8000 | 8000 | 8000 | 8000 |
| before removal, EU/mL | | | | | |
| Endotoxin concentration | 1.2 | 0.91 | 1.1 | 0.87 | 1.2 |
| after removal, EU/mL | | | | | |
| DNA recovery rate, % | 65 | 71 | 76 | 81 | 86 |

TABLE 5
Influence of different concentrations of human serum albumin
in protein sample on endotoxin removal from protein sample

| | Protein concentration in protein sample, mg/mL | | | |
|---|---|---|---|---|
| | 5 | 10 | 15 | 20 |
| Endotoxin concentration | 8000 | 8000 | 8000 | 8000 |
| before removal, EU/mL | | | | |
| Endotoxin concentration | 1.3 | 1.1 | 2.3 | 2.1 |
| after removal, EU/mL | | | | |
| Protein recovery rate, % | 93 | 91 | 94 | 90 |

It can be seen from the data in Tables 4 and 5 that the DNA or protein concentration has no influence on the removal of endotoxin. That is, whether the concentration of the biological product in the biological product sample from which endotoxin is intended to be removed is high or low, the endotoxin can be removed effectively by using the method according to the present invention, whereby the endotoxin concentration is reduced to meet the drug standards for use in clinical. Moreover, the loss of the biological product is little. Therefore, in the process of removing endotoxin from a biological product by using the method of the present invention, a wide range of concentrations of the biological product can be used.

### REFERENCES

1. Cai Huili. Endotoxin removal in separation and purification of biomedicines. Strait Pharmaceutical Journal 18(2), 2006, P157-9.
2. Magalhães P O. Methods of endotoxin removal from biological preparations: a review, J Pharm Pharm Sci. 2007 10(3):388-404
3. Daneshian M, Guenther A, Wendel A, Hartung T, and Von Aulock S. In vitro pyrogen test for toxic or immunomodulatory drugs. Journal of Immunological Methods 313:169-175.
4. Matt Cotton, et al., Lipopolysaccharide is a frequent contaminant of plasmid DNA preparations and can be toxic to primary human cells in the presence of adenovirus. Gene Therapy 1(4), 1994, 239-45.

### INDUSTRIAL APPLICABILITY

By using the kit provided in the present invention, the endotoxin in the biological product can be easily and quickly removed. The method for removing endotoxin has the advantages of easy operation and low cost, and has an endotoxin removal effect that is significantly superior to that of the conventional method, with the biological activity of the biological product being not affected. The endotoxin concentration in the biological product prepared by the method of the present invention meets the drug standards for use in clinical, and the loss of active substances is little. Therefore, the present invention is suitable for industrial application.

1.-26. (canceled)

27. A method for removing bacterial endotoxin from a biological product, comprising steps of: mixing the biological product containing endotoxin with an anionic surfactant solution, standing the resultant mixed solution, then adding a potassium salt or a potassium salt solution to precipitate the anionic surfactant and standing, and then centrifuging or filtering, to obtain a biological product solution from which the endotoxin has been removed.

28. The method according to claim 27, wherein the anionic surfactant is one or more of sodium dodecyl sulfate, sodium deoxycholate, sodium dodecyl sulfonate, sodium s-alkyl sulfates, sodium fatty alcohol polyoxyethylene ether sulfates, sodium oleyl sulfate, N-oleoyl poly(amino acid) sodium, sodium alkylbenzene sulfonates, sodium α-olefin sulfonates, sodium alkyl sulfonates, α-sulfo monocarboxylic acid esters, fatty acid sulfoalkyl esters, succinate sulfonate, alkyl naphthalene sulfonates, sodium alkane sulfoates, sodium ligninsulfonate, and sodium alkyl glyceryl ether sulfonates.

29. The method according to claim 27, wherein the potassium salt is one or more of potassium chloride, potassium acetate, potassium sulfate, potassium carbonate, potassium bicarbonate, potassium phosphate, potassium hydrogen phosphate, potassium dihydrogen phosphate, and potassium nitrate.

30. The method according to claim 28, wherein the anionic surfactant is sodium dodecyl sulfate, and/or sodium deoxycholate.

31. The method according to claim 29, wherein the potassium salt is potassium acetate, and/or potassium chloride, and the potassium salt solution is a potassium acetate solution and/or a potassium chloride solution.

32. The method according to claim 27, wherein the concentration of the anionic surfactant in the mixed solution is 0.1 wt % or higher.

33. The method according to claim 32, wherein the concentration of the anionic surfactant in the mixed solution is 0.1-10 wt %.

34. The method according to claim 28, wherein the concentration of the anionic surfactant in the mixed solution is 0.1-10 wt %.

35. The method according to claim 30, wherein the concentration of the anionic surfactant in the mixed solution is 0.1-10 wt %.

36. The method according to claim 27, wherein the final concentration of the potassium salt after being mixed with the mixed solution is higher than or equal to the concentration at which the anionic surfactant can be sufficiently precipitated.

37. The method according to claim 29, wherein the final concentration of the potassium salt after being mixed with the mixed solution is higher than or equal to the concentration at which the anionic surfactant can be sufficiently precipitated.

38. The method according to claim 31, wherein the final concentration of the potassium salt after being mixed with the mixed solution is higher than or equal to the concentration at which the anionic surfactant can be sufficiently precipitated.

39. The method according to claim 28, wherein when sodium dodecyl sulfate is used, the final concentration of the potassium salt is 0.3 M or higher.

40. The method according to claim 27, wherein the concentration of the anionic surfactant in the mixed solution is higher than that of the endotoxin.

41. The method according to claim 39, wherein the concentration of the anionic surfactant in the mixed solution is higher than that of the endotoxin.

42. The method according to claim 41, wherein the biological product is a protein or a nucleic acid.

43. A method for preparing an endotoxin-free biological product by using the method for removing bacterial endotoxin from a biological product according to claim 27, to obtain the biological product sample solution from which the endotoxin has been removed, and then separating the biological product from the biological product sample solution from which the endotoxin has been removed, to obtain an endotoxin-free biological product.

44. The method according to claim 43, wherein the biological product is a protein, the method comprises specifically the steps of:
1) adding the anionic surfactant to a protein containing endotoxin such that the final concentration of the anionic surfactant is 0.1 wt % or higher, uniformly mixing them to obtain a mixed solution, and standing the mixed solution for 5 min;
2) adding the potassium salt to the mixed solution obtained in Step 1) until no precipitate is produced, and mixing uniformly to obtain a mixed solution containing a precipitate; and standing the mixed solution for 5 min;
3) centrifuging the mixed solution containing a precipitate obtained in Step 2) at 14000 rpm for 5 min, discarding the precipitate, and collecting the supernatant; or filtering the mixed solution containing a precipitate obtained in Step 2) through a 0.45 μm PP membrane filter by centrifuging at 4000 rpm, to obtain a filtrate that is a protein sample solution from which the endotoxin has been removed; and
4) separating the protein from the protein sample solution with the removal of endotoxin obtained in Step 3) by precipitation or dialysis, to obtain an endotoxin-free protein.

45. The method according to claim 43, wherein the biological product is DNA, the method comprises specifically the steps of:
1) adding the anionic surfactant to DNA containing endotoxin such that the final concentration of the anionic surfactant is 0.1 wt % or higher, uniformly mixing them to obtain a mixed solution, and standing the mixed solution for 5 min;
2) adding the potassium salt to the mixed solution obtained in Step 1) until no precipitate is produced, and mixing uniformly to obtain a mixed solution containing a precipitate; and standing the mixed solution for 5 min;
3) centrifuging the mixed solution containing a precipitate obtained in Step 2) at 14000 rpm for 5 min, discarding the precipitate, and collecting the supernatant; or filtering the mixed solution containing a precipitate obtained in Step 2) through a 0.45 μm PP membrane filter by centrifuging at 4000 rpm, to obtain a filtrate that is a DNA sample solution from which the endotoxin has been removed; and
4) adding an equal volume of isopropanol to the DNA sample solution with the removal of endotoxin obtained in Step 3), mixing uniformly and standing for 30 min at room temperature, then centrifuging at 14000 rpm for 10 min, discarding the supernatant, and washing the precipitate with 70% ethanol; centrifuging at 14000 rpm for 10 min, discarding the supernatant, washing the precipitate and centrifuging once again, and removing ethanol by air drying, to obtain an endotoxin-free DNA.

46. A method for preparing an endotoxin-free biological product by using the method for removing bacterial endotoxin from a biological product according to claim 41, to obtain the biological product sample solution from which the endotoxin has been removed, and then separating the biological product from the biological product sample solution from which the endotoxin has been removed, to obtain an endotoxin-free biological product.
